(19) United States
(12) Patent Application Publication
(10) Pub. No.: US 2013/0165765 A1
(43) Pub. Date: Jun. 27, 2013

(54) PROPERTY INFORMATION ACQUIRING APPARATUS

(71) Applicant: CANON KABUSHIKI KAISHA, Tokyo (JP)

(72) Inventor: Hiroshi Nishihara, Kawasaki-shi (JP)

(73) Assignee: CANON KABUSHIKI KAISHA, Tokyo (JP)

(21) Appl. No.: 13/712,485

(22) Filed: Dec. 12, 2012

(30) Foreign Application Priority Data
Dec. 26, 2011 (JP) 2011-283687
Nov. 16, 2012 (JP) 2012-252353

(51) Int. Cl. A61B 6/00; A61B 5/00

(52) U.S. Cl. CPC A61B 6/5247; A61B 5/0035
USPC 600/407

(57) ABSTRACT

The present invention employs a property information acquiring apparatus comprising: a supporting unit configured to support a test subject and include an aperture into which a tested part of the test subject is inserted; a holding unit configured to hold the tested part inserted into the aperture; and a pressing unit that includes a receiving unit for receiving information relating to a property of the tested part and is pushed against a surface of the holding unit different from a surface for holding a test object, wherein the supporting unit includes a restricting part that restricts deformation caused in the holding unit by the pressing of the pressing unit.

### BACKGROUND OF THE INVENTION

1. Field of the Invention

The present invention relates to an apparatus that acquires information relating to the property of a test object.

2. Description of the Related Art

There are breast examination apparatuses (property information acquiring apparatuses) which include a bed for placing a test subject face down and in which a breast of the test subject is inserted into a hole provided to the bed. Among such breast examination apparatuses that utilize X-rays, there are some in which a breast of a test subject is held and compressed between a breast compression plate formed of a material transmissive to X-rays and an imaging plate including a sensor (Patent Literature 1: Japanese Translation of PCT Application No. H6-510930). In such breast examination apparatuses, the test subject inserts and hangs down the breast from a breast insertion opening provided to the bed that is a supporting platform. Then, imaging is performed by X-ray irradiation in a state where the hanging breast is clamped with the breast compression plate. This is because motion of the test subject at the time of imaging can be restricted and an accurate measurement can be achieved by performing the imaging in a state where the test subject is relaxed in a non-stressful position.

Also, a breast examination apparatus that includes a breast compression plate formed of a material transmissive to X-rays and ultrasound waves and obtains an X-ray image and an ultrasound echo image of a breast compressed by the breast compression plate is disclosed (Patent Literature 2: Japanese Translation of PCT Application No. H09-504211).

FIG. 5 shows the breast compression plate disclosed in Japanese Translation of PCT Application No. H09-504211. A breast compression plate 95 formed of the material transmissive to X-rays and ultrasound waves is provided with a metal reinforcement frame 96 for restricting warpage due to compression.

Meanwhile, there is a technique in which light of about 600 to 1500 nm wavelength having favorable transmissive properties for body tissues is used so that formation of new blood vessels and oxygen metabolism by hemoglobin along with the growth of tumor is measured and utilized for diagnosis based on the absorption properties of hemoglobin contained in blood with respect to the light. One such technique uses a photoacoustic effect. The photoacoustic effect is a phenomenon where irradiation of a substance with pulsed light in the order of nanoseconds causes the substance that has absorbed light energy in correspondence with the light absorption properties to momentarily expand and generate elastic waves. The elastic waves are detected and subjected to signal processing with an ultrasound transducer to obtain a receive signal. By mathematically performing analytical processing of the receive signal, sound pressure distribution of the elastic waves generated by the photoacoustic effect can be imaged. Because hemoglobin has a higher absorption rate of near-infrared light compared to water, fat, or protein that forms a body tissue, this is a suitable method for measurement of the new blood vessel or oxygen metabolism described above. Using the photoacoustic effect, clinical research for application in diagnosis of breast cancer or the like is strongly promoted.

There are cases where a breast compression plate such as that described above is provided also in a breast examination apparatus utilizing the photoacoustic effect. The purpose is to prevent a change in measurement position due to a breast moving during measurement and to obtain an image of a deep part by thinning the breast through compression. In the case where the breast compression plate is provided, elastic waves generated from a test object is received by an ultrasound transducer via the breast compression plate.

In the breast examination apparatus utilizing the photoacoustic effect, sound pressure distribution of the elastic waves due to the photoacoustic effect is imaged. In the case where the elastic wave is received in a state where the breast that is the test object is compressed by the compression plate as described above, the elastic wave may be received via the compression plate. Specifically, an ultrasound transducer is arranged to oppose the test object with the compression plate therebetween, and the elastic wave that has reached the ultrasound transducer via the compression plate is received with the ultrasound transducer. In this case, the thickness of the compression plate is preferably small so that attenuation of the elastic waves at the compression plate is restricted. However, when the thickness of the compression plate is reduced, there are cases where the compression plate is bent upon application of load on the compression plate. When a bend occurs in the compression plate in this manner, analytical processing in consideration of the bend becomes necessary upon generating a diagnostic image from the received elastic waves, thus causing a drawback such as longer time for image generation.

Also, the compression plate and a front surface portion of the ultrasound transducer are generally produced to have an acoustic impedance equal to that of a test object in consideration of an acoustic impedance mismatch. However, when a gap occurs between the compression plate and the ultrasound transducer due to distortion of the compression plate, air or the like enters this gap portion. Since the acoustic impedance of air does not match the acoustic impedance of the test object, a mismatch in the acoustic impedance occurs, causing a drawback in which the mismatch leads to attenuation of elastic waves and leads to low quality of a diagnostic image.

The present invention has been made in view of the problem described above, and an object thereof is to provide a technique for reducing deformation of a compression plate in an apparatus that acquires property information of a test object.

The present invention provides a property information acquiring apparatus comprising:

a supporting unit configured to support a test subject and include an aperture into which a tested part of the test subject is inserted;

a holding unit configured to hold the tested part inserted into the aperture; and

a pressing unit that includes a receiving unit for receiving information relating to a property of the tested part and is pushed against a surface of the holding unit different from a surface for holding a test object, wherein the supporting unit includes a restricting part that restricts deformation caused in the holding unit by the pressing of the pressing unit.

### SUMMARY OF THE INVENTION

With the present invention, a technique for reducing deformation of a compression plate in a property information acquiring apparatus can be provided.

Further features of the present invention will become apparent from the following description of exemplary embodiments with reference to the attached drawings.

### BRIEF DESCRIPTION OF THE DRAWINGS

FIG. 1A is a perspective view schematically showing a breast examination apparatus;

FIG. 1B is a partial sectional view schematically showing the breast examination apparatus;

FIG. 2A is a perspective view showing the configuration of a measuring unit;

FIG. 2B is a partial sectional view showing the configuration of the measuring unit;

FIGS. 3A and 3B are partially enlarged views each showing a region A of the measuring unit;

FIG. 4 is a configuration diagram of an ultrasound transducer unit; and

FIG. 5 is a configuration diagram of a breast compression plate of the related art.

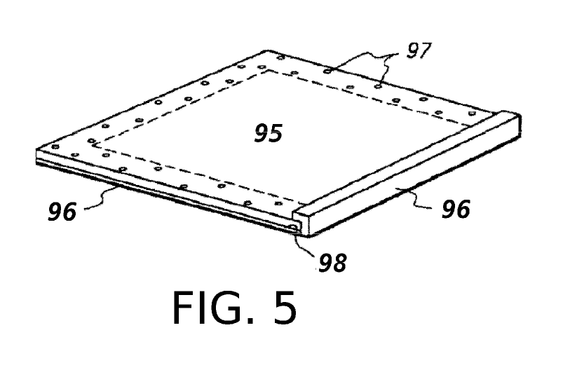

### DESCRIPTION OF THE EMBODIMENTS

A preferred embodiment of the present invention will be described below with reference to the drawings. Note that the dimension, material, and shape of the components described below as well as the relative arrangement thereof or the like are to be changed appropriately depending on various conditions or the configuration of an apparatus to which the present invention is applied, and are not intended to limit the scope of the invention to the description below.

A property information acquiring apparatus of the present invention includes an apparatus that transmits an elastic wave to a test object, receives a reflected wave reflected inside the test object, and acquires test object information as image data. Also, an apparatus utilizing a photoacoustic effect in which an elastic wave generated within a test object by irradiation of the test object with light (electromagnetic waves) is received and test object information is acquired as imaged data is included.

The test object information acquired in the case of the former is information that reflects the difference in acoustic impedance of tissues inside the test object. The test object information acquired in the case of the latter is source distribution of elastic waves caused by light irradiation, initial sound pressure distribution within the test object, light energy absorption density distribution or absorption coefficient distribution derived from the initial sound pressure distribution, or concentration distribution of substance forming a tissue. The concentration distribution of substance is, for example, oxygen saturation distribution or oxyhemoglobin and deoxyhemoglobin concentration distribution. The elastic wave referred to in the present invention is typically an ultrasound wave and is also called sound wave, ultrasound wave, or acoustic wave. The elastic wave generated by a photoacoustic effect is called photoacoustic wave or light-induced ultrasound wave. A transducer that is a receiving unit receives an elastic wave generated or reflected within a test object.

### EXAMPLE

In an example described below, a configuration example of a case where the present invention is applied to a breast examination apparatus utilizing a photoacoustic effect among property information acquiring apparatuses will be described. A test object at this time is a breast that is a part of a test subject body.

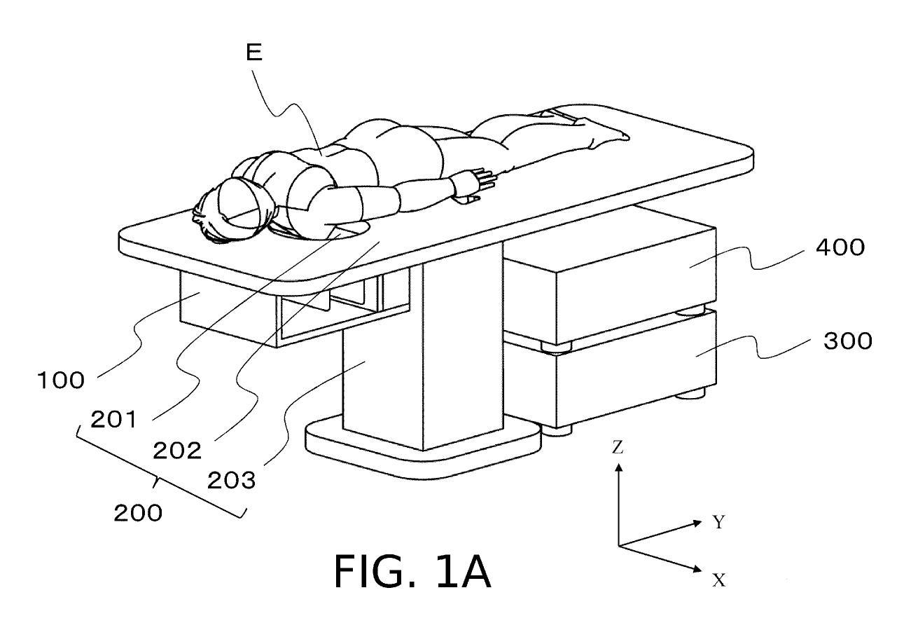

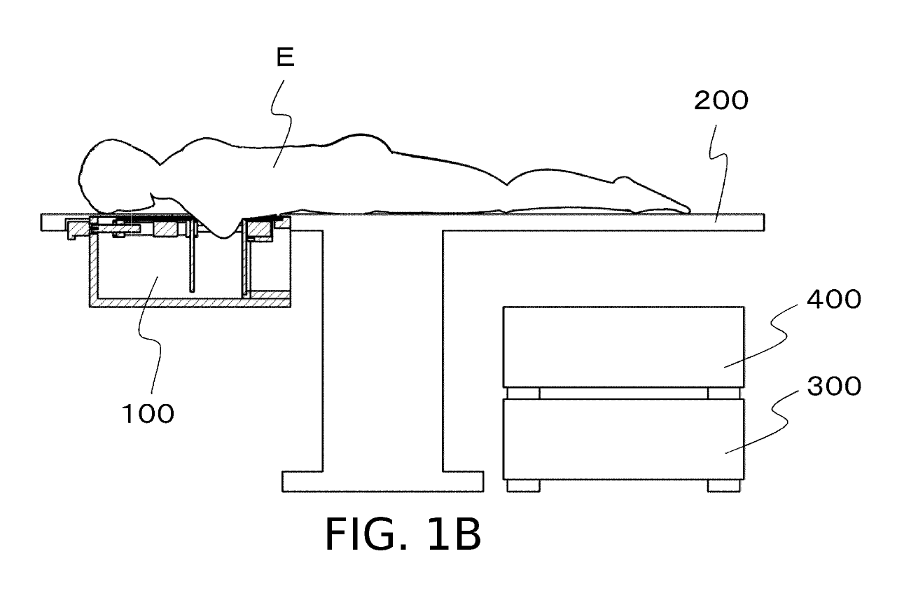

A schematic view of the breast examination apparatus utilizing a photoacoustic effect is shown in FIG. 1A and

FIG. 1B. FIG. 1A is a perspective view, and FIG. 1B is a partial sectional view when seen from the X-direction in FIG. 1A.

In FIG. 1A and FIG. 1B, reference numeral 100 denotes a measuring unit, 200 a bed unit, 300 a light source unit, 400 an electric unit, and E a test subject.

The measuring unit 100 is a device for measurement of a breast that is a test object of a test subject utilizing a photoacoustic effect in this example and, although details will be described later, includes a thoracic wall supporting plate that forms a supporting unit for supporting the test subject, an ultrasound transducer that is the receiving unit, and a compression plate that is a holding unit for holding a tested part.

The bed unit 200 is a device for placing the test subject E face down (in a prone position), is provided with a breast insertion opening 201 that is an aperture for inserting the breast as the tested part of the test subject, and includes a bed 202 that forms the supporting unit for supporting the test subject together with the thoracic wall supporting plate described above and a bed supporting column 203 that supports the bed 202.

The light source unit 300 that is a light irradiating unit for irradiating the breast as the tested part with light includes a laser light source that emits pulsed light of a particular wavelength in the order of nanoseconds which is to be irradiated onto the breast of the test subject E. Also, the light emitted from the laser light source is guided to the measuring unit 100 by a light guiding optical system such as an optical fiber, which is not shown. For the wavelength of the light emitted by the laser light source, a wavelength in accordance with the absorption spectrum of water, fat, protein, oxyhemoglobin, deoxyhemoglobin or the like forming body tissues is selected. As one example, a range of 600 to 1500 nm where light passes through in a favorable manner due to low absorption in water, which is the main component of internal body tissues, and the spectra of fat, oxyhemoglobin, and deoxyhemoglobin are distinctive is appropriate. As a specific example, it is favorable to form the laser light source with a semiconductor laser, a wavelength-tunable laser, or the like capable of emitting a plurality of light lasers having different wavelengths.

The electric unit 400 includes a power supply part that supplies power to the measuring unit 100 and the light source unit 300, a control device that controls these units, and a signal processing device that processes a signal measured with the measuring unit 100. The signal processing device performs imaging of the sound pressure distribution of elastic waves (acoustic waves) generated by a photoacoustic effect.

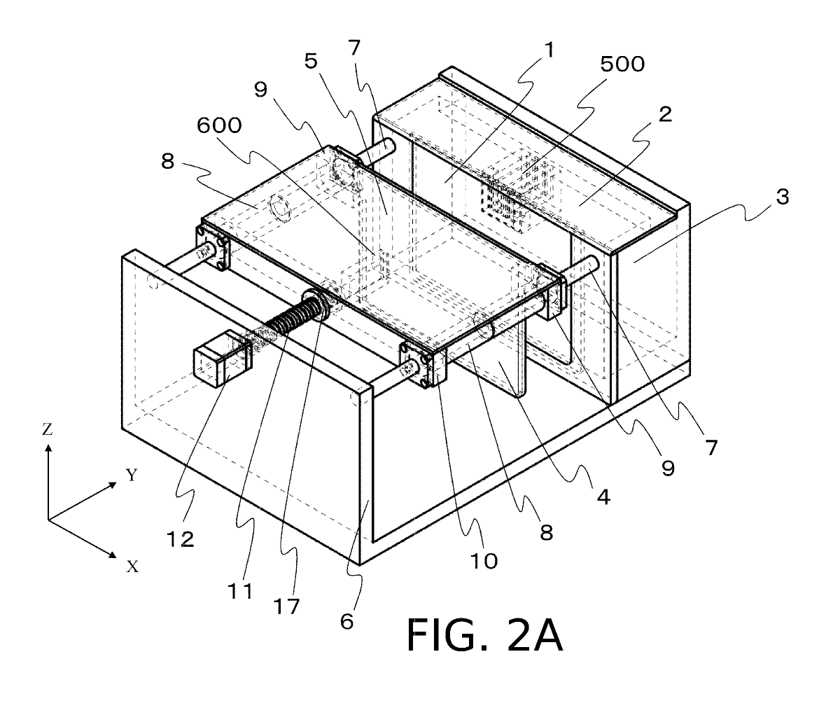

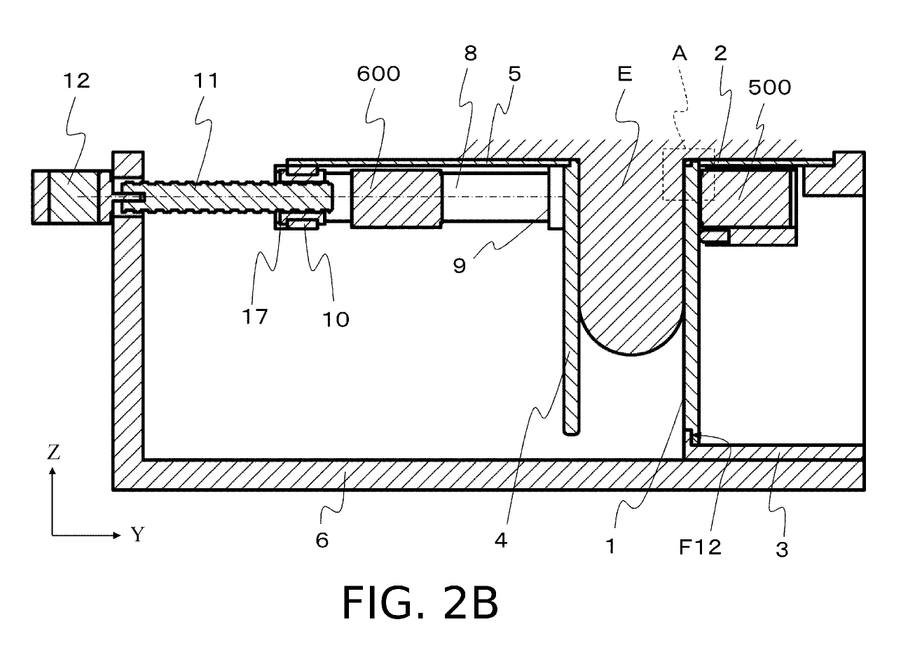

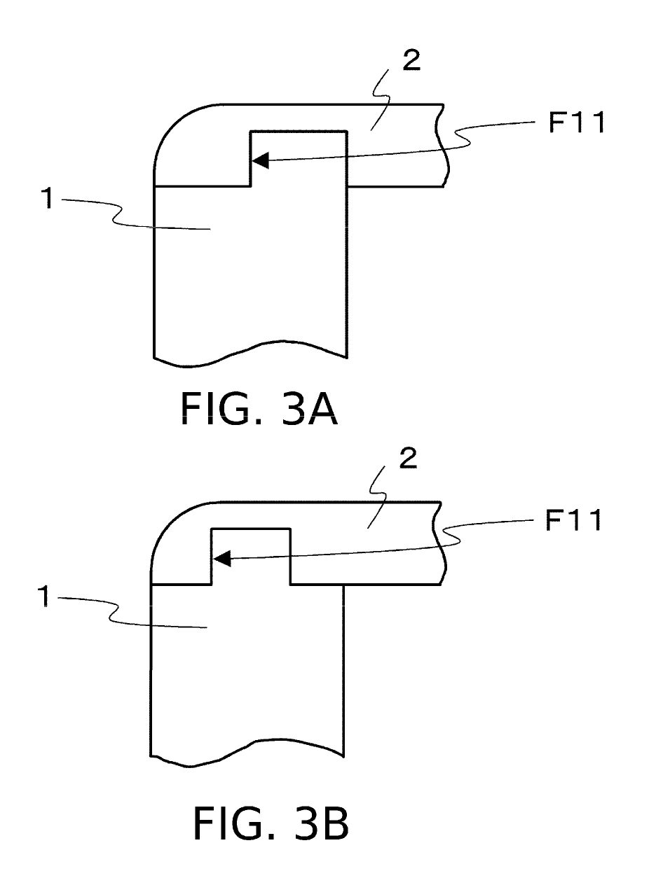

A configuration diagram of the measuring unit 100 is shown in FIG. 2A, FIG. 2B, and FIG. 3A. FIG. 2A is a perspective view, FIG. 2B is a partial sectional view when seen from the X-direction in FIG. 2A, and FIG. 3A is a partially enlarged view of a region A in FIG. 2B.

A first compression plate 1 with which the caudal side (foot side) of the breast of the test subject E is held while being compressed and a first thoracic wall supporting plate 2 that supports a thoracic wall near and below a bust are attached to a first compression plate supporting base 3. The first compression plate 1 is in contact with the first thoracic wall supporting plate 2 at one edge, i.e., an upper edge herein.

Also, an ultrasound transducer unit 500 including an ultrasound transducer 13 (not shown) that is the receiving unit for receiving an acoustic wave generated at the breast as the tested part through irradiation of the breast as the tested part with light by the light source unit 300 as the light irradiating unit is attached firmly to the first compression plate 1 with an acoustic matching agent therebetween. Also, the ultrasound transducer unit 500 is caused to scan in the X-direction and Z-direction (within the XZ plane) in FIG. 2A with a scanning mechanism, which is not shown.

A second compression plate 4 (second holding unit) with which the cranial side of the breast of the test subject E is held while being compressed and a second thoracic wall supporting plate 5 that supports the thoracic wall on the cranial side are attached to a slide mechanism that moves in the Y-direction in FIG. 2A. The slide mechanism is configured of two main shafts 7 fixed to the first compression plate supporting base 3 and a second compression plate supporting base 6, a bearing 8 that is guided by the main shaft 7 to slide, and a first bearing housing 9 and a second bearing housing 10 that hold the bearing 8. Also, the second bearing housing 10 is provided with a nut 17, and the second compression plate 4 is movable in the Y-direction in FIG. 2A by rotating a screw 11 with a motor 12. On the other hand, the first compression plate 1 is fixed and does not move.

In this embodiment, a mechanism that compresses and holds the breast is configured of the first compression plate 1 (first holding unit), the second compression plate 4 (second holding unit), and the slide mechanism. The purpose of compressing and holding the breast with two compression plates in this manner is to prevent a change in measurement position due to the breast moving during measurement and to enable imaging of a deep part by thinning the breast through compression. Note that, of the first compression plate 1 and the second compression plate 4 that are a pair of plate-like members, the second compression plate 4 that is one of the plate-like members is movable to change the interval between the first compression plate 1 and the second compression plate 4 that are the pair of plate-like members. Note that although the breast that is the tested part is compressed and held in this embodiment, compression is not necessary in the case where it suffices to hold the breast that is the tested part such that the measurement position does not change. In this case, depending on the shape or arrangement of the first compression plate (first holding unit), the slide mechanism or the second compression plate may become unnecessary.

Also, an illuminating unit 600 that guides a light laser emitted from the light source unit 300 to the breast is provided. The illuminating unit 600 is caused to scan in the X-direction and Z-direction (within the XZ plane) in FIG. 2A by a scanning mechanism, which is not shown, in synchronization with the driven ultrasound transducer unit 500.

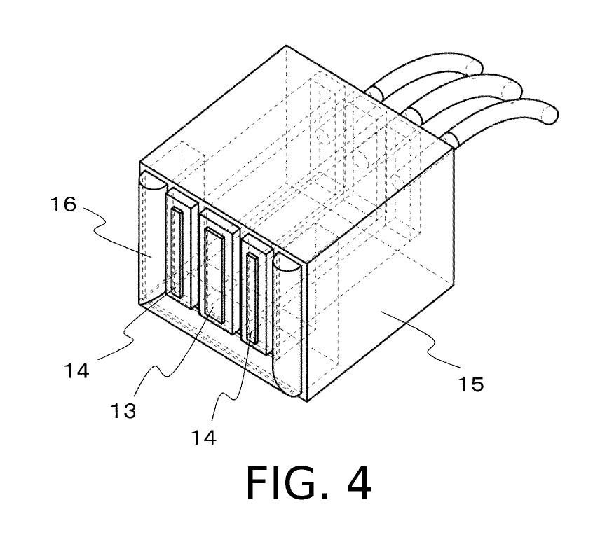

FIG. 4 is a configuration diagram of the ultrasound transducer unit 500. The ultrasound transducer unit 500 has a housing 15, and the ultrasound transducer 13 that is the receiving unit and an illuminating optical system 14 are attached to the housing 15. Also, the housing 15 is provided with a sealing member 16 for holding the acoustic matching agent between the first compression plate 1 and the ultrasound transducer 13. The ultrasound transducer unit 500 corresponds to a pressing unit of the present invention.

Details of the respective components will be described below.

In this embodiment, the ultrasound transducer 13 that is the receiving unit is arranged to oppose the breast that is the tested part with the first compression plate 1 that is the first holding unit therebetween. Thus, it is preferable that the material of the first compression plate 1 have high transmissive properties and low attenuation properties with respect to elastic waves (acoustic waves) generated by a photoacoustic effect and have high transmissive properties and low attenuation properties with respect to light emitted by the laser light source. Examples of such material include silica glass, polymethylpentene polymer, polycarbonate, and acrylic resin.

The acoustic matching agent of ultrasound gel or liquid is filled between the first compression plate 1 and the ultrasound transducer 13. It is preferable that the acoustic matching agent also have high transmissive properties and low attenuation properties with respect to elastic waves generated by a photoacoustic effect and have high transmissive properties and low attenuation properties with respect to light emitted by the laser light source. An example of the matching agent is water, castor oil, ultrasonography gel, polyethylene glycol, or the like.

Also, the sealing member 16 is provided in order to hold the acoustic matching agent in a favorable manner. The sealing member 16 is configured of an elastic material such as rubber or resin and pushed against the first compression plate 1 with appropriate load.

The ultrasound transducer unit 500 described above has a configuration in which the acoustic matching agent is held by the sealing member 16. However, the ultrasound transducer 13 may be pressed against the first compression plate 1 applied with the acoustic matching agent such that the two members are firmly in contact.

The second compression plate 4 that is the second holding unit is a flat plate having high transmissive properties and low attenuation properties with respect to light emitted by the laser light source. Examples of the material forming the second compression plate 4 include glass, polymethylpentene polymer, polycarbonate, and acrylic resin. Note that the properties desired for the respective compression plates change depending on the arrangement of the transducer at either one of the first and second compression plates and the arrangement of the laser light source at either one (or both).

The first thoracic wall supporting plate 2 is provided between the thoracic wall of the test subject E and the ultrasound transducer unit 500. Also, the second thoracic wall supporting plate 5 is provided between the thoracic wall of the test subject E and the illuminating unit 600. By providing the first and second thoracic wall supporting plates, the combination of the respective first and second thoracic wall supporting plates enables the rigidity to be increased and the breast or the test subject to be held safely. Further, with the presence of the first thoracic wall supporting plate 2 and the second thoracic wall supporting plate 5, a portion (thoracic wall or the like) in the vicinity of the breast (tested part) of the test subject is supported such that body tissues (for example, skin near the rib or collarbone, subcutaneous fat, muscle, or the like) not held by the two compression plates are prevented from hanging down outside the compression plates under the influence of gravity. As a result, narrowing of a scan range due to the thoracic wall hanging down by gravity to interfere with the ultrasound transducer unit 500 or the illuminating unit 600 can be avoided in the prone position-type property information acquiring apparatus as in this example.

The acoustic matching agent of gel or liquid is continuously supplied by a pump, which is not shown, to space formed by the first compression plate 1 and the sealing member 16. As shown in FIG. 4, the sealing member 16 has a structure in which the upper edge of the ultrasound transducer unit 500 is open and the other three edges are sealed. In order to constantly fill the acoustic matching agent between the first compression plate 1 and the ultrasound transducer 13, the acoustic matching agent is caused to overflow from the open upper edge of the sealing member 16.

The sealing member 16 is produced by molding of rubber, resin, or the like melted at high temperature. The shape of the sealing member 16 produced with such a method may vary in a range of about plus or minus 0.1 mm. Therefore, in order for the sealing member 16 to firmly contact the first compression plate 1 without a gap, it is necessary to cause deformation in the sealing member 16 formed of the elastic material by pushing the sealing member 16 against the first compression plate 1. With this pushing force, pressing load is applied with respect to the first compression plate, thus posing a risk of deformation. Although details will be described later, such deformation has a possibility of adversely affecting accurate measurement, and thus the deformation needs to be reduced as much as possible.

Thus, in the present invention, the first thoracic wall supporting plate 2 that is a first supporting unit includes a restricting part in order to reduce the deformation amount of warpage, bend, or the like of the first compression plate 1 due to pressing load with which the sealing member 16 is pushed against the first compression plate 1. Specifically, the first thoracic wall supporting plate 2 that is the first holding unit includes a portion for holding the first compression plate 1 that is the first holding unit. (Hereinafter, this portion is referred to as a first pressing load supporting part F11 that regulates the movement of the first compression plate 1 to the tested part side). The first pressing load supporting part corresponds to a first restricting part of the present invention.

In FIG. 3A, the first thoracic wall supporting plate 2 is provided with the first pressing load supporting part F11 formed of a surface approximately perpendicular to the pressing load. The first pressing load supporting part F11 is a depressed portion provided to the first thoracic wall supporting plate 2, and more specifically is a side surface located nearer the test object among side surfaces on the inside of the depressed portion. Note that the depressed portion provided to the first thoracic wall supporting plate 2 is formed to fit with a protruded portion provided to the first compression plate 1. The first pressing load supporting part F11 contacts one wall surface of the protruded portion extending at an upper edge where the first thoracic wall supporting plate 2 is in contact among edges of the first compression plate 1. By providing such a surface, it is possible to restrict deformation and confine the deformation amount even if the first compression plate 1 receives the pressing load to the breast side due to pressing, since the first thoracic wall supporting plate 2 is integrated to increase rigidity.

The pressing load referred to herein is the load in the horizontal direction (Y direction in FIG. 2A) in the drawing with respect to the compression plate due to the sealing member 16 being pushed against the first compression plate 1. Therefore, the surface approximately perpendicular to the pressing load refers to a surface (XZ plane in FIG. 2A) in the vertical direction shown as F11 in the drawing. By attaching the first compression plate 1 to the first pressing load supporting part F11, the first thoracic wall supporting plate 2 functions as a reinforcement member that reduces the bend amount of the first compression plate 1.

For attachment of the first compression plate 1 to the first pressing load supporting part F11, joining can be done with a thread or an adhesive. Also, without adhesion of the first compression plate 1 and the first thoracic wall supporting plate 2, the two may be merely fitted. Alternatively, a slight gap that causes the first compression plate 1 to contact the first pressing load supporting part F11 at the time of bend due to the pressing load may be provided for attachment.

Further, in order to further reduce the deformation amount of the first compression plate 1, a member that supports a lower part of the first compression plate 1 may be provided with a second pressing load supporting part that regulates the movement of the first compression plate 1. The second pressing load support section corresponds to a second restricting part (regulating part) of the present invention. That is, as shown in FIG. 2B, the first compression plate supporting base 3 that supports the first compression plate 1 from below is provided with a second pressing load supporting part F12 formed of a surface (XZ plane in FIG. 2A)approximately perpendicular to the pressing load. By attaching the first compression plate 1 to the second pressing load supporting part F12, the first compression plate supporting base 3 functions as a reinforcement member that reduces the deformation amount of warpage, bend, or the like of the first compression plate 1.

For attachment of the first compression plate 1 to the second pressing load supporting part F12, joining using a thread or an adhesive is favorable. Herein, the first compression plate supporting base 3 corresponds to a supporting base of the present invention.

Also, as the material forming the first thoracic wall supporting plate 2 and the second thoracic wall supporting plate 5, a material with a large Young's modulus is preferable. Metal, a metal compound, or the like may be suitably used. Examples of such material include, iron, stainless steel, and tungsten carbide. Particularly, tungsten carbide having a Young's modulus about twice that of iron is one of the preferable materials. Also, stainless steel that is advantageous in terms of shapability and strength is also one of the preferable materials.

As a specific example, the pressing load in the case where deformation of about 0.2 mm is caused by pushing the sealing member 16 formed of fluorocarbon rubber against the first compression plate 1 is about 20 N. At this time, the first compression plate 1 is configured of polymethylpentene polymer with a thickness of 7 mm, the first thoracic wall supporting plate 2 of tungsten carbide with a thickness of 3 mm, and the first compression plate supporting base 3 of aluminum. In this configuration, the first pressing load supporting part F11 with a width of 3 mm and the second pressing load supporting part F12 with a width of 10 mm are provided in order to apply the present invention. At this time, bend in the case where load of 20 N is applied with respect to the first compression plate 1 at a position of the ultrasound transducer unit 500 is calculated to be about 0.06 mm at maximum.

On the other hand, bend of the first compression plate 1 in the case where the present invention is not applied and two edges of the first compression plate 1 parallel to the Z-axis are supported is calculated in a similar manner to be about 0.4 mm at maximum.

In the case where the sealing member 16 is pushed against the first compression plate 1 in a position where the bend is maximum to cause a deformation of 0.2 mm and firm contact, scanning with the ultrasound transducer unit 500 on the supporting part side of the first compression plate 1 where the bend is small causes a further a deformation of 0.4 mm at maximum in the sealing member 16. In the case where the load with respect to the deformation of the sealing member 16 changes linearly, the pressing load that occurs at a contact surface of the first compression plate 1 and the sealing member 16 changes in a range of 20 to 60 N. Therefore, it is difficult to perform scanning in a stable manner due to a large fluctuation in load at the time of scanning with the ultrasound transducer unit 500.

Also, since the motor used in the scanning needs to be driven even under the maximum pressing load, that with a large output torque is to be used, thus resulting in large noise and high cost. Also, in the case where the sealing member 16 is pushed against the first compression plate 1 in the vicinity of the supporting part of the first compression plate 1 to cause a deformation of 0.2 mm and firm contact, scanning with the ultrasound transducer unit 500 in the X-direction causes the deformation amount of the sealing member 16 to be reduced by the degree of bend in the first compression plate 1. Therefore, a gap is formed at the contact surface of the first compression plate 1 and the sealing member 16, and holding the matching agent appropriately is not possible.

Since the deformation of the first compression plate 1 is 0.06 mm at maximum in the example in which the present invention is applied, the range of fluctuation in the pressing load can be made to be 20 to 20.6 N. Therefore, since the fluctuation of load at the time of scanning with the ultrasound transducer unit 500 can be reduced, scanning can be performed in a stable manner. Also, since the first compression plate 1 and the sealing member 16 can be firmly in contact even in the case where there is variance of about plus or minus 0.1 mm in the shape of the sealing member 16, the matching agent can be held appropriately.

FIG. 3B is a view showing a modification example of the first pressing load supporting part. In this modification example, the position of the protruded portion of the first compression plate to be fitted with the first pressing load supporting part is different. A wall surface of the protruded portion nearer the ultrasound transducer is not integrated with a wall surface of a main body of the first compression plate, but is in a position nearer the test object. In this case as well, the deformation amount of the first compression plate can be reduced by the first pressing load supporting part fitted with the protruded portion of the first compression plate regulating the movement of the first compression plate under pressing load.

Also, the first and second pressing load supporting parts are approximately perpendicular with respect to the XY plane in the example described above. However, in reality, this is not limiting. For example, the angle of mesh at the time of fitting may be made steeper by causing the surface forming the first pressing load supporting part to be inclined within a range of 90 degrees or less in a counterclockwise direction in FIGS. 3A and 3B.

Also, the first and second pressing load supporting parts are shown in this example to be supporting parts having a planar shape, but may also be a spherical surface or a cylindrical surface. In other words, a configuration is acceptable as long as the pressing load supporting part regulates the movement of the first compression plate to the test object side when pushed against by the ultrasound transducer unit 500.

For the material forming the first compression plate supporting base 3, the second compression plate supporting base 6, the first bearing housing 9, and the second bearing housing 10, it is favorable to use aluminum, iron, stainless steel, or the like.

The main shaft 7 is configured of a member having a cylindrical shape and of which the surface of steel material has undergone a hardening process. It is favorable to form the bearing 8 with a linear bushing, a solid bearing, or the like that can slide smoothly even under the weight of the test subject E. Also, it is favorable to form the screw 11 and the nut provided to the second bearing housing 10 with a ball screw that can be driven with low friction. Also, for the motor 12, a DC motor, an AC motor, a stepper motor, or the like may be used.

The ultrasound transducer 13 is configured of a piezoelectric element having a piezoelectric effect that converts a change in pressure due to the received elastic wave into an electrical signal, and a plurality of the piezoelectric elements are arranged to be approximately rectangular as shown in FIG. 4. It is known that the formation of new blood vessels along with the growth of tumor such as cancer increases in the case where the size of the tumor is 2 to 3 mm or greater. Therefore, for the piezoelectric element, a piezoelectric ceramic material as represented by lead zirconate titanate (PZT) suited for detection of elastic waves of 0.5 MHz to several tens of megahertz generated from a light absorber of several millimeters or less due to a photoacoustic effect may be used. Also, piezoelectric polymer film material or the like as represented by polyvinylidene fluoride (PVDF) may be used. The ultrasound transducer 13 is connected to the signal processing device of the electric unit 400 by a cable.

The illuminating optical system 14 is configured of a bundle of a plurality of optical fibers. A light emitting end of the optical fibers is made to be approximately rectangular as shown in FIG. 4 by adjusting the arrangement of the optical fibers. Also, for an illuminating optical system of the illuminating unit 600, that equivalent to the illuminating optical system 14 described above is used.

In the breast examination apparatus in this example, as described above, rigidity with respect to pressing load of the first compression plate (breast compression plate) that is the holding unit can be increased to reduce (restrict) deformation such as warpage or bend by providing the respective first and second restricting parts (pressing load supporting parts) to the first and second thoracic wall supporting plates. Since the first compression plate can be thinned as a result, attenuation of the elastic waves propagating through the plate is reduced, making it possible to receive a signal with high signal-to-noise ratio.

Further, reduced deformation of the first compression plate causes various effects. One of the effects is faster image generation for the inside of the test object. That is, upon imaging of the sound pressure distribution of elastic waves due to a photoacoustic effect, analytical processing for the degree of deformation in the compression plate is necessary in the case where deformation of the first compression plate is large. However, if the deformation is small, the degree of deformation can be ignored in analytical processing, and therefore image can be generated at high speed.

Another effect is a more appropriate usage amount of the acoustic matching agent. If the deformation of the first compression plate is small, controlling the filled amount of the acoustic matching agent used between the first compression plate and the ultrasound transducer is easy. That is, when the deformation amount is large, a region where a firm contact at the contact surface of the sealing member of the ultrasound transducer and the first compression plate cannot be sufficiently maintained occurs, and the acoustic matching agent cannot be held appropriately. As a result, elastic waves generated by a photoacoustic effect cannot be detected accurately. On the other hand, when the deformation amount is small, the acoustic matching agent can be held in a favorable manner to maintain accuracy in a photoacoustic measurement even if pressing load is applied at the time of scanning with the ultrasound transducer along the surface of the first compression plate.

Still another effect is a reduction of load on the slide mechanism in the case where the ultrasound transducer is caused to scan along the surface of the first compression plate. In the scanning, the friction properties between the sealing member and the first compression plate and the friction force caused by the pressing load with which the sealing member is pushed determine the load on the slide mechanism. When the first compression plate is bent by the pressing load at this time, the sealing member in firm contact with the first compression plate is also deformed in a similar manner. When the bend amount of the first compression plate at a scanning surface differs depending on the location, load at the time of scanning fluctuates. On the other hand, when the deformation amount is small, load can be made even and restricted.

Also, since the first thoracic wall supporting plate and the second thoracic wall supporting plate are provided in the present invention, body tissues not held between the two compression plates are supported. As a result, the hanging body tissues do not interfere with the ultrasound transducer, and it is possible to obtain an image near the thoracic wall of the test object.

Note that although the present invention has been described with the above example of the breast examination apparatus that receives an acoustic wave generated by light irradiation to acquire property information of a test object, this is not limiting. An X-ray irradiation-type mammography or the like using an X-ray irradiating unit for irradiating a tested part with an X-ray and a receiving unit for receiving the X-ray which has been irradiated onto the tested part by the X-ray irradiating unit is also one form to which the present invention may be applied.

Also, although the present invention has been described with the above example of a prone position-type measuring apparatus, this is not limiting. The present invention may be applied also to a standing position-type test object information acquiring apparatus.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No.2011-283687, filed on Dec. 26, 2011, and, Japanese Patent Application No.2012-252353, filed on Nov. 16, 2012, which are hereby incorporated by reference herein in their entirety.

What is claimed is:

1. A property information acquiring apparatus comprising:
a supporting unit configured to support a test subject and include an aperture into which a tested part of the test subject is inserted;
a holding unit configured to hold the tested part inserted into the aperture; and
a pressing unit that includes a receiving unit for receiving information relating to a property of the tested part and is pushed against a surface of the holding unit different from a surface for holding a test object, wherein
the supporting unit includes a restricting part that restricts deformation caused in the holding unit by the pressing of the pressing unit.

2. The property information acquiring apparatus according to claim 1, wherein the restricting part is a portion, of the supporting unit, that holds the holding unit.

3. The property information acquiring apparatus according to claim 1, wherein the supporting unit includes a bed.

4. The property information acquiring apparatus according to claim 1, further comprising a light irradiating unit configured to irradiate the tested part with light, wherein the receiving unit receives an acoustic wave generated at the tested part by the light irradiating unit irradiating the tested part with light.

5. The property information acquiring apparatus according to claim 4, wherein the receiving unit is arranged to oppose the tested part with the holding unit therebetween, and the holding unit is polymethylpentene.

6. The property information acquiring apparatus according to claim 1, further comprising an X-ray irradiating unit configured to irradiate the tested part with an X-ray,
wherein the receiving unit receives the X-ray which has been irradiated onto the tested part by the X-ray irradiating unit.

7. The property information acquiring apparatus according to claim 1, wherein the supporting unit is made of metal or a metal compound.

8. The property information acquiring apparatus according to claim 7, wherein the supporting unit is made of tungsten carbide.

9. The property information acquiring apparatus according to claim 7, wherein the supporting unit is made of stainless steel.

10. The property information acquiring apparatus according to claim 1, wherein the holding unit includes a pair of plate-like members arranged to oppose each other with the test object therebetween, and one of the pair of plate-like members is movable to change an interval between the pair of plate-like members.

11. The property information acquiring apparatus according to claim 1, wherein the holding unit includes a protruded portion extending at a portion in contact with the supporting unit, and the restricting part is a depressed portion that fits with the protruded portion.
